(19) United States
(12) Patent Application Publication
(10) Pub. No.: US 2009/0155860 A1
(43) Pub. Date: Jun. 18, 2009

(54) Process for the Production of Oligosaccharides

(76) Inventors: Athanasios K. Goulas, Reading Berkshire (GB); Georgios Tzortzis, Reading Berkshire (GB)

Correspondence Address: CHRISTIE, PARKER & HALE, LLP, PO BOX 7068, PASADENA, CA, 91109-7068, US

(21) Appl. No.: 12/084,681

(22) PCT Filed: Nov. 2, 2006
(86) PCT No.: PCT/EP2006/068029

§ 371 Date: May 6, 2008

(30) Foreign Application Priority Data
Nov. 8, 2005 (GB) 0522740.0

(51) Int. Cl. C12P 19/14

(52) U.S. Cl. 435/101

(57) ABSTRACT

A process for producing a prebiotic mixture of galactooligosaccharides from lactose using galactosidase producing bacteria, wherein the bacterial cells may be reused in synthesis reactions without loss of yield of the product.

### CROSS-REFERENCE TO RELATED APPLICATION(S)

This application is a National Phase Patent Application of International Application Number PCT/EP2006/068029, filed on Nov. 2, 2006, which claims priority of British Patent Application Number 0522740.0, filed on Nov. 8, 2005.

The present invention relates to a process for producing a prebiotic mixture of galactooligosaccharides.

### SUMMARY OF THE INVENTION

According to the invention there is provided a process for synthesising a galactooligosaccharide mixture comprising disaccharide Gal (α1-6)-Gal, at least one trisaccharide selected from Gal (β1-6)-Gal (β1-4) Glc, Gal (β1-3)-Gal (β1-4)-Glc, tetrasaccharide Gal (β1-6)-Gal (β1-6)-Gal (β1-4)-Glc and pentasaccharide Gal (β1-6)-Gal (β1-6)-Gal (β1-6)-Gal (β1-4)-Glc, where Gal represents a galactose residue and Glc represents a glucose residue wherein a culture of Bifidobacterium bifidum cells is added to lactose or a lactose-containing substrate and the bacterial cells are reused in up to eight consecutive synthesis reactions without loss of yield of the galactooligosaccharide mixture.

After 8 synthesis reactions a slight decrease in the produced oligosaccharides occurs, which after 12 times of re-use accounts for 10% of the total products formed in the initial reaction.

### BRIEF DESCRIPTION OF THE DRAWINGS

FIG. 1 shows a typical time course during the production of GOS by samples as analyzed by HPLC.

FIG. 2 is a HPAEC-PAD chromatogram of a GOS mixture synthesized by b. bifidum NCIMB 41171.

### DETAILED DESCRIPTION OF THE INVENTION

A prebiotic is defined as a non-digestible food ingredient that beneficially affects a mammalian host by selectively stimulating the growth and/or activity of one or a limited number of bacteria in the colon, thereby resulting in an improvement in the health of the host.

Galactooligosaccharides are non-digestible carbohydrates, which are resistant to mammalian gastrointestinal digestive enzymes but are fermented by specific colonic bacteria. They have been shown to have very good prebiotic activity in the proximal and transverse parts of the colon.

GB 2 412 380 describes a noval strain of Bifidobacterium bifidum capable of producing a galactosidase enzyme activity that converts lactose to a novel mixture of galactooligosaccharides comprising Gal (α 1-6)-Gal, Gal (β 1-6)-Gal (β 1-4) Glc, Gal (β 1-3)-Gal (β 1-4)-Glc, Gal (β 1-6)-Gal (β 1-6)-Gal (β 1-4)-Glc and Gal (β 1-6)-Gal (β 1-6)-Gal (β 1-6)-Gal (β 1-4)-Glc. The strain was deposited under accession number NCIMB 41171 at the National Collection of Industrial and Marine Bacteria, Aberdeen on 31 Mar. 2003.

Such a deposited strain of Bifidobacterium bifidum, or its biologically functional equivalent, can be used to produce the galactooligosaccharide mixture, as defined above, in the process of the present invention. The phrase “biologically functional equivalent” is contoured to mean a strain of Bifidobacterium bifidum that produces a galactosidase enzyme activity that converts lactose into the mixture of galactooligosaccharides as defined above.

In order to produce the mixture of galactooligosaccharides as defined above lactose or a lactose-containing substrate is treated with a strain of Bifidobacterium bifidum as defined above.

A suitable lactose-containing substrate may be selected from commercially available lactose, whole milk, semi-skimmed milk, skimmed milk, whey and fat-filled milk. Such milk products may be obtained from cows, buffalos, sheep or goats. Fat-filled milk is defined as whole milk that has been skimmed to remove the dairy fat, which is subsequently replaced by the addition of vegetable fat or oil.

It has been found that the majority of galactosidases produced by the deposited strain of Bifidobacterium bifidum are cell bound making it possible to use the whole cells for the synthesis of the galactooligosaccharide mixture. It has been found unexpectedly that the bacterial cells (biomass) can be recovered by centrifugation and re-used in consecutive synthesis reactions up to 8 times without significant loss of biomass or changes in reaction times whilst yielding the same amounts of product oligosaccharides.

The present invention will be further described by way of reference to the following example.

### EXAMPLE

### Materials and Methods

All chemicals and media preparations used throughout this study were from Sigma (Dorset, UK), VWR (Dorset, UK), and Oxoid (Basingstoke, UK).

### Microorganism Growth and Enzyme Production

Bifidobacterium bifidum NCIMB 41171 was isolated from a human faecal sample. The working culture was propagated in broth containing tryptone 15 g/l, Lab Lemco (conventional meat extract) 2.5 g/l, yeast extract 7.5 g/l, K₂HPO₄ 4.5 g/l, cysteine-HCl 0.05 g/l, lactose 2.5 g/l, glucose 7.5 g/l and Tween 80 1 ml/l. The pH of the growth medium was adjusted to 6.7 before autoclaving and incubations were carried out under anaerobic conditions (10:10:80; H₂:CO₂:N₂) at 37° C.

Fermentations for B. bifidum enzyme production were performed in 7 and 150 L fermentation vessels taking all the necessary precautions to ensure aseptic operation. The culture media used for maximum enzyme production contained tryptone 7.5 g/l, Lab Lemco (conventional meat extract) 7.5 g/l, yeast extract 7.5 g/l, K₂HPO₄ 2 g/l, cysteine-HCl 0.5 g/l, lactose 4 g/l, glucose 6 g/l and Tween 80 0.5 ml/l. Oxygen-free conditions in the fermenters were achieved by flushing the culture media with oxygen-free nitrogen during the cooling period after sterilisation and also by creating a nitrogen blanket above the culture during growth. Inoculum levels were at 5% (v v⁻¹), the temperature was maintained at 37° C., stirring at 100 rpm, and the pH was regulated at 6.7 using sodium hydroxide solutions (2M).

More than two-thirds of the galactosidase activity produced by B. bifidum NCIMB 41171 was observed to be bound on the cell-wall of the microorganism and the remainder was secreted in the culture supernatant. For this reason, and due to ease of biomass collection by centrifugation (at 7,000×g), the galactosidase enzyme bound to the microorganism cells was collected and used as the enzyme preparation for GOS synthesis. To assist biomass collection, the culture pH (regulated at 6.7 during most of the exponential growth phase) was allowed to drop during the stationary stage of growth to a value between 5 and 5.5 that induced cell flocculation.

The collected cell pellet was re-suspended in 0.1 M phosphate buffer (pH 6.8), washed twice and subsequently treated with toluene. Treatment of B. bifidum biomass with toluene, according to Onishi, Yamashiro and Yokozeki, Appl. & Env. Microbiol. (1995), 61 (11), 4002-4025, increased cell permeability and thus the observed galactosidase activities. This treatment was performed by re-suspending the cells, collected from 11 culture, in 80 ml 0.1 M phosphate buffer (pH 6.8) and adding 0.16 ml of toluene to this suspension. This preparation was placed in a shaking water bath at 20° C. for 1 h. The cells were then washed three times with buffer, frozen and freeze dried. This freeze dried biomass preparation was used for GOS synthesis.

Biomass monitoring during fermentations was carried out by the weight of cells retained on 0.2 μm filters after washing with deionised water and drying for 4 h at 105° C. Bacterial numbers were monitored by plating on a Wilkins-Chalgreen Anaerobe agar.

### Determination of α- and β-Galactosidase Activity, pH and Temperature Optimum Determination

Determination of the β-galactosidase activity contained in the B. bifidum biomass was performed using 4-nitrophenyl-β-D-galactopyranoside as substrate, in 0.1 M phosphate buffered solutions (pH 6.8) at 40° C. Disodium tetraborate (0.2 M) was used to stop the enzymatic reaction and develop the colour. Enzyme activity was measured as a function of the liberated O-nitrophenol determined by absorbance at 420 nm. Corrections for substrate and biomass interferences were taken into account. One unit of β-galactosidase was defined as the amount of enzyme liberating 1 μmole of O-nitrophenol per min at the above specified conditions.

The pH optimum for β-galactosidase activity in the B. bifidum cells was determined by performing enzyme activity measurements (as described above) of a standard biomass preparation at different pH values (between 4 and 8). Solutions of 10 mM 2-nitrophenyl-β-D-galactopyranoside were prepared using 0.1 M phosphate and citrate-phosphate buffers that were arranged at the desirable pH.

The temperature optimum for the β-gal activity contained in the B. bifidum cells was determined by performing enzyme activity measurements (as described above) of a standard biomass preparation at different temperatures between 30 to 55° C.

α-Galactosidase activity was determined and defined in the same manner as the beta but using as substrate 4-nitrophenyl-α-D-galactopyranoside.

### GOS Synthesis and By-Product Inhibition

Synthesis of GOS was performed using pure lactose and ultrafiltration cheese whey permeate solutions.

When pure lactose was used as substrate (450, 500 mg/ml), synthesis was performed in 0.1 M phosphate (pH 6.8) and 0.1 M citric acid/sodium citrate (6.2) buffered solutions, at 40±0.5° C., stirring at 100 rpm. After lactose was dissolved and temperature equilibrated at 40° C., 2.5 g of freeze-dried enzyme (344 U g⁻¹) were added per 100 ml of synthesis mixture. Reactions were followed over a 24 h period. Samples were boiled for 10 min to inactivate the enzyme and consequently analysed for their carbohydrate content. Higher lactose synthesis concentrations were not applicable due to the crystallisation of lactose observed when the temperature was reduced to 40° C.

Under the above mentioned GOS synthesis conditions (at 450 mg/ml substrate concentration) optimum oligosaccharide concentration was observed at a time period of 6 h. In order to test the possibility of re-using the same biomass for repeated synthesis reactions, an experiment was performed where repeated 450 mg/ml synthesis reactions were performed using the same biomass which was collected by centrifugation at 7,000 rpm. A series of 12 consecutive 6 h synthesis reactions were performed over a 6 day period with the biomass being stored at 2-4° C. during the in-between time intervals. Samples for carbohydrate analysis were collected after centrifugation to avoid reducing biomass concentration.

Concentrated whey ultrafiltration permeate (in powder form) was kindly supplied by Volac International Ltd (Liverpool, UK). The preparation provided contained 0-0.5% (w/w) fat, 4.5-7.5% protein, 8-10% ash, 82% lactose and a pH value when diluted in water between 5-5.5. Before synthesis, all preparations of whey permeate were heated at 95° C. to dissolve the crystallised lactose and centrifuged for 10 min at 7,000 rpm to remove the precipitate observed as a result of heat denaturation of peptides present. This precipitate accounted for 2.6% (w/w) of the total solution weight under the conditions used for its removal. Elimination of this proteinous precipitate was considered necessary in order to be able to collect the B. bifidum biomass by centrifugation and re-use it for subsequent synthesis reactions. Synthesis conditions and enzyme concentration were as described for the pure lactose synthesis reactions.

In order to test the effect of glucose and galactose on the GOS production a series of experiments were performed, where simultaneously with lactose (400 mg/ml) as the substrate, varying concentrations of glucose and galactose (100 or 150 mg/ml) were added initially in the reaction mixture. These experiments were performed at pH 6.8 (0.1 M phosphate buffer), at 40±0.5° C., stirring at 100 rpm and 2.5 g of freeze-dried biomass (344 U/g) were added per 100 ml of synthesis mixture).

All the above GOS synthesis reactions were performed in duplicate.

Selective Removal of Monosaccharides from GOS Mixtures

Selective purification of the above produced oligosaccharides from the monosaccharides generated in the mixture was attempted by yeast fermentation. The strain Saccharomyces cerevisiae was used, due to the selective fermentation characteristics that it shows towards different sugars. Glucose and galactose were monosaccharide by-products during GOS synthesis, formed by lactose hydrolysis and galactose transfer to water molecules acting as trans-galactosylation acceptors.

Purification of the oligosaccharides produced during this study and of a commercial oligosaccharide mixture (Vivinal GOS, from Borculo Domo Ingredients, Zwolle, Holland; 57% (w w⁻¹) GOS, 23% lactose, 22% glucose and 0.8% galactose) was carried out. Solutions of the carbohydrate mixtures at a sugar concentration of 450 mg/ml were prepared in 0.1 M phosphate buffer (pH 6.8), in order to maintain a pH appropriate for yeast metabolism, and filter-sterilised. Fermentations took place in shaking flasks at 30° C. with the addition of 1 g of freeze-dried yeast (29×10⁹ cfu g⁻¹) per 100 ml of solution. Fermentations were followed over a period of 32 h and samples were analysed for their carbohydrate ethanol and protein content. Yeast cell enumeration was performed on CM129 Tryptone Soya agar plates. All GOS purification fermentations were performed in duplicate.

Sample Analysis for their Carbohydrate and Ethanol Content.

Synthesis and yeast fermentation samples were analysed by high performance liquid chromatography (HPLC) using an Aminex HPX-87C Ca⁺² resin-based column (300×7.7 mm) supplied by Bio-Rad Laboratories Ltd (Hertfordshire, U.K.) and an HPLC analyser coupled to a refractive index detector. The column was maintained at 85° C. and HPLC grade water was used as mobile phase at a flow rate 0.6 ml min⁻¹. Under these conditions oligosaccharides eluted as two not well resolved peaks followed by disaccharides (one peak) and monosaccharides where glucose and galactose appeared as separate peaks. Ethanol determination with a standard calibration curve was possible using this column since it eluted separately.

Quantitative determination of the oligosaccharides (degree of polymerisation (DP)≧3), disaccharides, and monosaccharides was performed by using standard calibration curves of maltotriose, lactose, glucose and galactose respectively.

In order to quantify the amount of transgalactosylated disaccharides contained in the combined peak of disaccharides, as determined by the HPLC analysis, synthesis samples were also analysed by high performance anion-exchange chromatography coupled with pulsed amperometric detection (HPAEC-PAD). A pellicular anion-exchange resin based column CarboPac PA-1 from Dionex Chromatography (Surrey, UK) was used. Carbohydrates were eluted at 1 ml/min flow rate using gradient mobile phase concentrations of sodium hydroxide and sodium acetate solutions at 20±0.5° C. Lactose, in this case, eluted as a separate peak allowing its quantitative determination by using a standard calibration curve which, in combination with the HPLC data, allowed quantitative determination of the transgalactosylated disaccharides.

Selected samples were further analysed by gas chromatography mass spectrometry after derivatisation to sugar oximes using hydroxylamine chloride in pyridine and persilylation using hexamethyldisilazane and trifluoroacetic acid. The column used during the analysis was the DB-17MS (length 30 m, I.D. 0.25 mm, Film 0.25 μm) from J&W Scientific (USA).

### Results and Discussion

Fermentation for the Production of B. bifidum NCIMB 41171 Galactosidase

During the fermentations for the production of the B. bifidum NCIMB 41171 an exponential growth phase of 7-8 h was observed with bacterial numbers rising from 13×10⁶ to 43×10⁸ cfu ml⁻¹. A freeze-dried biomass content of 2.68 g L⁻¹ at the beginning of the stationary phase was measured. Maximum galactosidase activity was observed when the culture was well in the stationary phase showing a β-galactosidase activity of 1 U ml⁻¹ of culture (supernatant plus cells). This eventually would give an activity of 205.5 U g⁻¹ of freeze-dried biomass. The α-galactosidase activity of this preparation was determined to be 3.05 U g⁻¹. Reproducibility between the 7 L and the pilot plant (150 L) fermentations was very good and this biomass was treated with toluene, frozen, freeze-dried and subsequently used for all synthesis reactions. Freezing and freeze-drying of the B. bifidum NCIMB 41171 biomass did not affect galactosidase activity but it affected the viability of the bacteria which was of no concern for the intended use. Treatment of the B. bifidum cells with toluene, before freeze-drying, increased cell permeability which resulted to an increase on the α- and β-galactosidase activities observed to 5.04 and 344 U g⁻¹ respectively.

### Synthesis of GOS

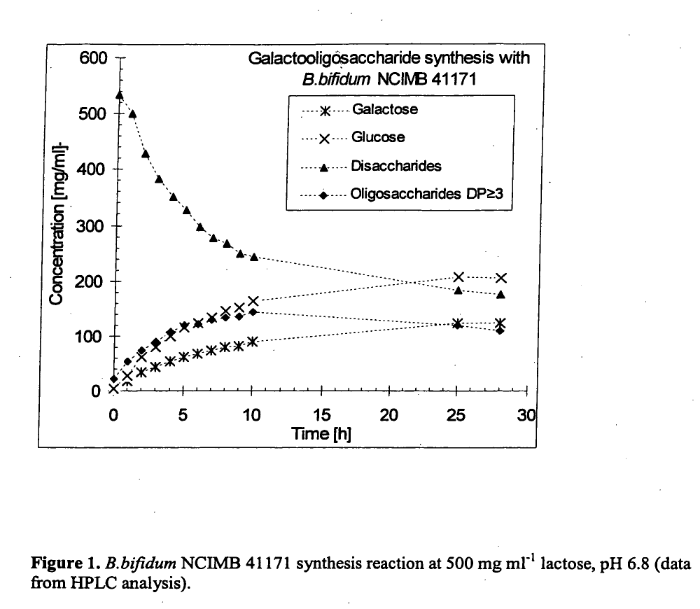

Synthesis of GOS was performed using the cell-bound enzymes of B. bifidum NCIMB 41171. More than one galactosidase is present in B. bifidum strains and the oligosaccharides produced, during this study, were considered a product of their combined activity. FIG. 1 shows a typical time course during the production of GOS by samples as analysed by HPLC. Oligosaccharide concentration increased initially to a maximum and subsequently decreased when transgalactosylation activity became less pronounced than the hydrolytic activity. Substantial amounts of glucose and galactose were formed from lactose hydrolysis.

Oligosaccharide concentrations increased with increasing lactose concentration since the water activity of the synthesis solutions decreases as substrate concentration increases making the transfer reaction of galactose to water molecules less likely to occur. In table 1 the carbohydrate compositions are shown from synthesis reactions at the maximum possible substrate concentrations at pH 6.8, 6.2 and using as lactose source whey permeate powder. As can be seen, the amounts of transgalactosylated disaccharides (disaccharides other than lactose) present in the mixtures were very close to the concentrations of the higher degree of polymerisation (DP≧3) oligosaccharides produced. Increased amounts of hydrolysis products were observed as the pH of the synthesis decreased from 6.8 to 6.2 and 5.4 when whey permeate powder was used as substrate. Fixing the reaction pH of the whey permeate substrates at higher values proved to be undesirable due to the presence of peptides and amino acids which gave extensive Maillard browning at increased pH.

Lactose conversion at maximum oligosaccharide concentration was determined (table1) using the actual lactose concentrations measured by HPAEC-PAD and the highest oligosaccharide concentration was observed at around 80 to 85% lactose conversion. As the lactose concentration used for synthesis increased the substrate conversion values where the maximum oligosaccharide concentration was observed also increased. The yields of oligosaccharides varied between 39 and 43% when pure lactose was used as the substrate and between 36 and 38% when whey permeate was the lactose source. There was no significant difference observed in the yield values between different initial substrate concentrations.

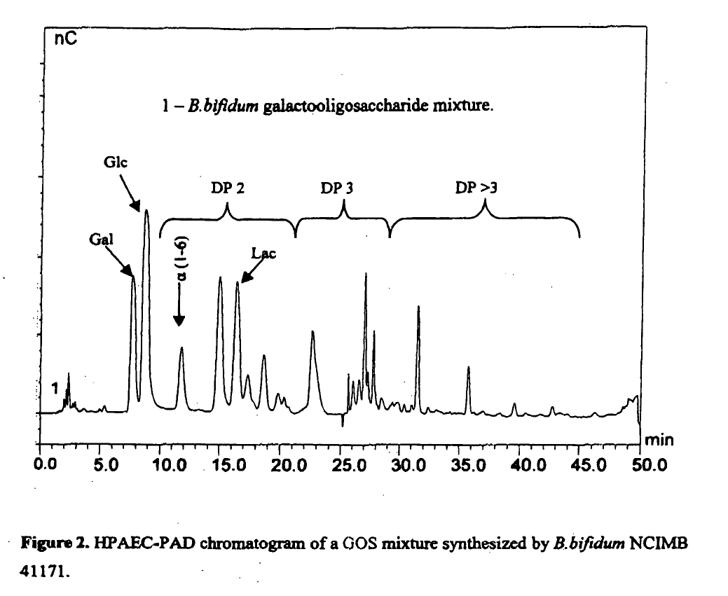

In FIG. 2 a representative HPAEC-PAD chromatogram is shown of the oligosaccharide mixtures produced. A variety of different GOS were produced in decreasing amounts as the molecular weight of the carbohydrates increased. A significant finding was a disaccharide that eluted at the same retention time with an α(1-6) galactobiose standard. For confirming this result samples were analysed by gas chromatography mass spectrometry after derivatisation to their sugar oximes. Again the presence of the α-linked disaccharide was confirmed by the presence of two well resolved peaks with retention times 27.7 and 29.0 minutes under the specified analysis conditions. Comparison of the main spectra ratios of each peak yielded very small differences between the standard and the synthesis samples confirming again the presence of this carbohydrate. In the experiment where the possibility of reusing the B. bifidum biomass for consecutive synthesis reactions was tested, the same amount of biomass was successfully reused in 8 subsequent 450 mg/ml (lactose) synthesis reactions yielding the same amounts of product oligosaccharides (as shown in table 1) at similar time periods of reaction.

From this point onwards a slight decrease in the produced oligosaccharides was observed which, after 12 times of re-use, accounted for 10% of the total products formed in the initial reactions.

TABLE 1
Carbohydrate composition of synthesis reactions at 450 and 500 mg ml-1
initial lactose concentration at maximum galactooligosaccharide concentration.

| | Synthesis | GOS | GOS | | | | Substrate |
|---|---|---|---|---|---|---|---|
| | Init. Subst. | DP ≧ 3 | DP 2 | Lac. | Glc | Gal | Conversion |
| | Concentration (mg/ml) | | | | | | % (−) |
| Phosphate | 450 | 94.03 | 99.17 | 79.29 | 120.45 | 57.06 | 82.38 |
| buf. pH 6.8 | 500 | 109.02 | 88.43 | 72.77 | 154.16 | 75.62 | 85.45 |
| Citrate | 450 | 85.59 | 111.67 | 80.77 | 109.52 | 62.46 | 82.05 |
| buf. pH 6.2 | 500 | 94.66 | 115.79 | 79.84 | 132.98 | 76.73 | 84.03 |
| Whey | 450 | 77.40 | 85.05 | 82.34 | 124.8 | 80.42 | 81.4 |
| permeate | 500 | 89.8 | 99.34 | 80.17 | 140.64 | 90.05 | 85.97 |

*Substrate conversions at maximum oligosaccharide concentration were calculated based on the lactose concentrations determined by HPAEC-PAD.

1. A process for synthesising a galactooligosaccharide mixture comprising disaccharide Gal (α1-6)-Gal, at least one trisaccharide selected from Gal (β1-6)-Gal (β1-4) Glc, Gal (β1-3)-Gal (β31-4)-Glc, tetrasaccharide Gal (β1-6)-Gal (β1-6)-Gal (β1-4) Glc and pentasaccharide Gal (β1-6)-Gal (β1-6)-Gal (β1-6)-Gal (β1-4)-Glc, where Gal represents a galactose residue and Glc represents a glucose residue wherein a culture of Bifidobacterium bifidum cells is added to lactose or a lactose-containing substrate characterised in that said cells are reused in up to eight consecutive synthesis reactions without loss of yield of said galactooligosaccharide mixture.

2. The process according to claim 1, wherein said culture of B. bifidum is a culture of strain NCIMB 41171 deposited with the National Collection of Industrial and Marine Bacteria, Aberdeen, UK on 31 Mar. 2003, or a biologically functional equivalent as defined herein.

3. The process according to claim 1 or claim 2, wherein the lactose-containing substrate is selected from the group consisting of whole milk, semi-skimmed milk, skimmed milk, whey and fat-filled milk.

4. The process according to claim 3, wherein the milk is obtained from cattle, buffalos, sheep or goats.
